(19) United States
(12) Patent Application Publication
(10) Pub. No.: US 2023/0324305 A1
(43) Pub. Date: Oct. 12, 2023

(54) MEASUREMENTS OF TEMPORAL DYNAMICS USING OPTICAL SCATTERING IN A DIFFUSIVE CAVITY

(71) Applicant: University of Central Florida Research Foundation, Inc., Orlando, FL (US)

(72) Inventors: Aristide Dogariu, Orlando, FL (US); Ruitao Wu, Orlando, FL (US)

(21) Appl. No.: 18/133,914

(22) Filed: Apr. 12, 2023

Related U.S. Application Data
(60) Provisional application No. 63/330,186, filed on Apr. 12, 2022.

(51) Int. Cl. G01N 21/75; G01N 21/47; B01L 3/00; G01N 33/50

(52) U.S. Cl. CPC G01N 21/75; G01N 21/4738; B01L 3/502715; G01N 33/5008; G01N 2201/08; G01N 2201/065; B01L 2300/168

(57) ABSTRACT

A measurement system may include a diffusive cavity including a reflective internal surface and one or more ports, a sample chamber located within the diffusive cavity, and a light source to direct measurement light into the diffusive cavity through one of the one or more ports of the diffusive cavity. The diffusive cavity may uniform illumination of the sample through diffusive reflection of at least one of the measurement light from the light source or scattered measurement light from the sample. The system may further include two or more detectors to capture light exiting at least one of the one or more ports. The system may further include a controller to receive detection signals from the two or more detectors indicative of the scattering of the measurement light by the sample and determine one or more time-varying properties of the sample based on the detection signals.

### CROSS-REFERENCE TO RELATED APPLICATION

The present application claims the benefit under 35 U.S.C. § 119(e) of U.S. Provisional Application Ser. No. 63/330,186 filed Apr. 12, 2022, entitled MEASUREMENTS OF TEMPORAL DYNAMICS USING OPTICAL SCATTERING IN A DIFFUSIVE CAVITY, naming Aristide Dogariu and Ruitao Wu as inventors, which is incorporated herein by reference in the entirety.

### TECHNICAL FIELD

The present disclosure relates generally to measurements of dynamic light scattering and, more particularly, to measurements of dynamic light scattering in a multiple scattering regime.

### BACKGROUND

Measurements of the dynamics of dynamic media are useful for a wide range of applications including, but not limited to, biological and material sciences. Optical scattering techniques are well-suited for such measurements since time-varying properties of a dynamic medium are encoded in fluctuations of scattered intensity of incident light. However, extraction of the time-varying properties of the dynamic medium from optical scattering is an inverse problem that presents various challenges for wide applicability of the technique. For example, this inverse problem may be simplified for certain extreme scattering conditions such as the deterministic single scattering regime, but enforcement of these conditions may severely limit the general applicability of the technique. As another example, the inverse problem may be solved in more dynamic scattering conditions if additional information about the optical path lengths in the dynamic medium are known. However, analytical solutions for the optical path lengths only exist under limited experimental conditions such that this approach may also have limited applicability. There is therefore a need to develop systems and methods to cure the above deficiencies.

### SUMMARY

A measurement system is disclosed, in accordance with one or more illustrative embodiments of the present disclosure. In one illustrative embodiment, the system includes a diffusive cavity including a reflective internal surface, where the diffusive cavity includes one or more ports. In another illustrative embodiment, the system includes a sample chamber located within the diffusive cavity, where the sample chamber is configured to hold a sample. In another illustrative embodiment, the system includes a light source configured to direct measurement light into the diffusive cavity through one of the one or more ports of the diffusive cavity, where the diffusive cavity provides uniform illumination of the sample through diffusive reflection of at least one of the measurement light from the light source or scattered measurement light from the sample. In another illustrative embodiment, the system includes one or more detectors configured to capture light exiting at least one of the one or more ports. In another illustrative embodiment, the system includes a controller to receive detection signals from the one or more detectors indicative of the scattering of the measurement light by the sample and determine one or more time-varying properties of the sample based on the detection signals.

A measurement method is disclosed in accordance with one or more illustrative embodiments of the present disclosure. In one illustrative embodiment, the method includes directing measurement light into a diffusive cavity through one of one or more ports of the diffusive cavity, where the diffusive cavity includes a reflective internal surface, and where the diffusive cavity provides uniform illumination of a sample in the diffusive cavity through diffusive reflection of at least one of the measurement light or scattered measurement light from the sample. In another illustrative embodiment, the method includes capturing light exiting at least one of the one or more ports of the diffusive cavity with one or more detectors. In another illustrative embodiment, the method includes receiving detection signals from the one or more detectors indicative of the scattering of the measurement light by the sample. In another illustrative embodiment, the method includes determining one or more time-varying properties of the sample based on the detection signals.

It is to be understood that both the foregoing general description and the following detailed description are exemplary and explanatory only and are not necessarily restrictive of the invention as claimed. The accompanying drawings, which are incorporated in and constitute a part of the specification, illustrate embodiments of the invention and together with the general description, serve to explain the principles of the invention.

### BRIEF DESCRIPTION OF DRAWINGS

The numerous advantages of the disclosure may be better understood by those skilled in the art by reference to the accompanying figures.

FIG. 1A is a conceptual diagram of a dynamic scattering measurement system, in accordance with one or more embodiments of the present disclosure.

FIG. 1B is a conceptual schematic of the dynamic scattering measurement system including two detectors distributed to receive light from two ports of a diffusive cavity, in accordance with one or more embodiments of the present disclosure.

FIG. 1C is a conceptual schematic of the dynamic scattering measurement system including two detectors distributed to receive light from a single port of a diffusive cavity, in accordance with one or more embodiments of the present disclosure.

FIG. 1D is a is a conceptual schematic of the dynamic scattering measurement system including a tubular sample chamber extending between ports of the diffusive cavity, in accordance with one or more embodiments of the present disclosure.

FIG. 2 is a plot of measured field-field correlation function in the strong scattering regime of light-matter interaction, in accordance with one or more embodiments of the present disclosure.

FIG. 3 is a conceptual plot of a log-log representation of ratio between the microscopic and perceived correlation times for different regimes of light-matter interaction, in accordance with one or more embodiments of the present disclosure.

FIG. 4 includes conceptual schematics of diffusive walks under various conditions, in accordance with one or more embodiments of the present disclosure.

FIG. 5A is a conceptual view of the dynamic scattering measurement system used for the generation of the data in FIGS. 5B-10, in accordance with one or more embodiments of the present disclosure.

FIG. 5B is a plot of the temporal correlation function of signals from the two detectors coupled to the diffusive cavity of FIG. 5A, in accordance with one or more embodiments of the present disclosure.

FIG. 6 is a plot of a comparison between measured mean path and a predicted mean path for dynamic media with similar microscopic but different macroscopic properties, in accordance with one or more embodiments of the present disclosure.

FIG. 7A includes a plot of the ratio τ₀/τₘ for different experimental geometries, in accordance with one or more embodiments of the present disclosure.

FIG. 7B includes a plot of the values of mean optical path-length of light, in accordance with one or more embodiments of the present disclosure.

FIG. 8 is a plot of the diffusion coefficient of colloidal media measured inside the diffusive cavity, in accordance with one or more embodiments of the present disclosure.

FIG. 9 includes various plots providing a comparison of measurements taken at different geometries over different range of concentrations, in accordance with one or more embodiments of the present disclosure.

FIG. 10 includes a plot of experimentally-measured cross-correlation for samples having varying particle sizes, in accordance with one or more embodiments of the present disclosure.

FIG. 11 is a flow diagram illustrating steps performed in a method for measuring time-varying properties of a dynamic medium, in accordance with one or more embodiments of the present disclosure.

### DETAILED DESCRIPTION

Reference will now be made in detail to the subject matter disclosed, which is illustrated in the accompanying drawings. The present disclosure has been particularly shown and described with respect to certain embodiments and specific features thereof. The embodiments set forth herein are taken to be illustrative rather than limiting. It should be readily apparent to those of ordinary skill in the art that various changes and modifications in form and detail may be made without departing from the spirit and scope of the disclosure.

Embodiments of the present disclosure are directed to systems and methods for measurements of time-varying properties of a dynamic medium based on optical scattering in a diffusive cavity to ensure strong coupling between incident light and the sample with uniform illumination to ensure diffusive scattering conditions.

In a diffusive scattering regime with strong light-matter coupling (e.g., conditions with a relatively large reduced scattering coefficient (μₛ′)), a relationship between intrinsic dynamics of a dynamic medium and a perceived timescale of scattered light by the dynamic medium is generally determined by the distribution of photon path lengths in the dynamic medium (e.g., a probability density function of the optical path length (P(s)). Further, this distribution of photon path lengths generally depends on microscopic characteristics of scattering events, macroscopic properties of the dynamic medium, and the particular measurement configuration.

However, it is contemplated herein that there exists a generalized invariance property (e.g., a generalized Cauchy invariant property for dynamic media) for diffusive scattering regimes providing that a ratio between the intrinsic dynamics of a dynamic medium and the perceived timescale of scattered intensity fluctuations depends on macroscopic properties of the dynamic medium and the experimental conditions rather than on detailed microscopic properties or geometry-dependent light-matter interactions. For example, a field-field correlation of scattering signals from two detectors may be accurately approximated by a first moment of a mean optical path length ( [symbol] s [symbol] ) for short correlation times, which may be determined by macroscopic properties such as the volume and surface area of the dynamic medium during a measurement. Since such macroscopic properties are easily measured and/or controlled, measurements of the dynamic properties of arbitrarily complex dynamic media may be performed so long as diffusive scattering conditions are maintained. In some embodiments, various macroscale properties are tunable during a measurement to provide additional flexibility for ensuring that diffusive scattering conditions are maintained.

It is further contemplated herein that enclosing a sample of a dynamic medium to be measured in a diffusive cavity (herein referred to as a Cauchy cavity, an integrating sphere, or the like) may ensure the conditions necessary for diffusive scattering. Further, the use of the diffusive cavity may ensure a diffusive scattering regime regardless of the microscopic properties of the dynamic sample (or at least ensure a diffusive scattering regime for a wide range of dynamic samples). As a result, the systems and methods disclosed herein may be suitable for measurements of time-varying properties of arbitrarily complex dynamic media under practical experimental conditions.

It is additionally contemplated herein that the systems and methods disclosed herein utilizing a diffusive cavity for scattering measurements may further enable highly sensitive measurements and/or measurements of low-volume/low-concentration samples. In particular, the diffusive cavity may reflect scattered light back to the sample to provide multiple scattering events and thus increased interaction between measurement light and the sample, which may increase the signal to noise ratio of the measurement relative to traditional techniques without such a diffusive cavity.

Some embodiments of the present disclosure are directed to measurement systems for dynamic samples including a coherent light source, a diffusive cavity, at least two detectors, and a controller to determine time-varying properties of the dynamic sample based on signals from the detectors.

Measurements of time-varying properties of dynamic samples are generally described in Wu, Ruitao, and Aristide Dogariu, “Dynamics of complex systems in Cauchy cavities,” Physical Review A 105.4 (2022): 043523; which is incorporated herein by reference in its entirety.

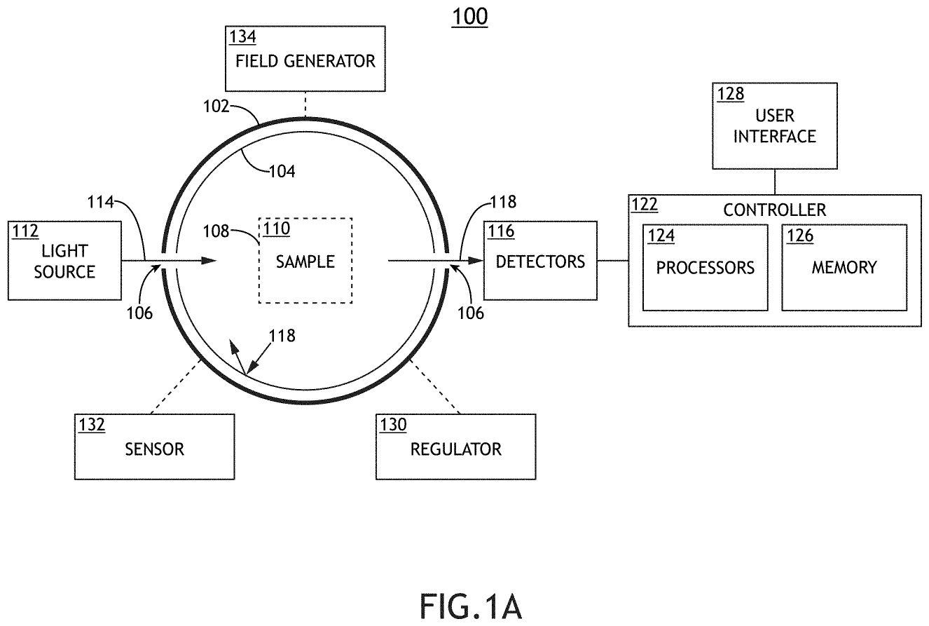

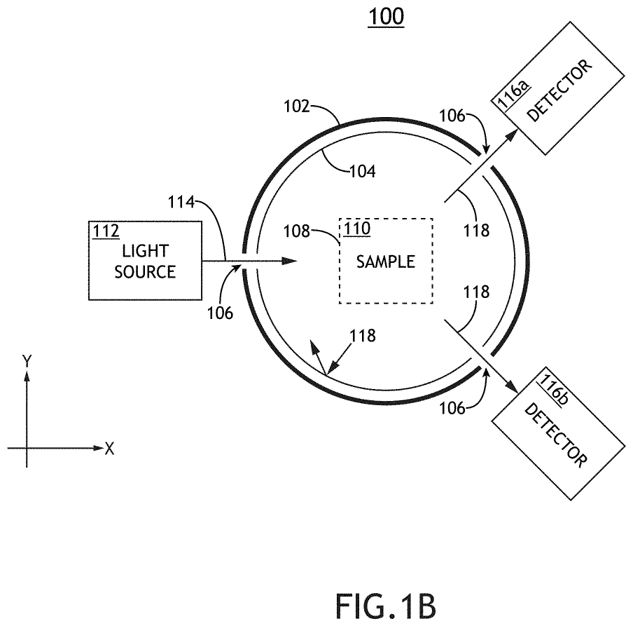

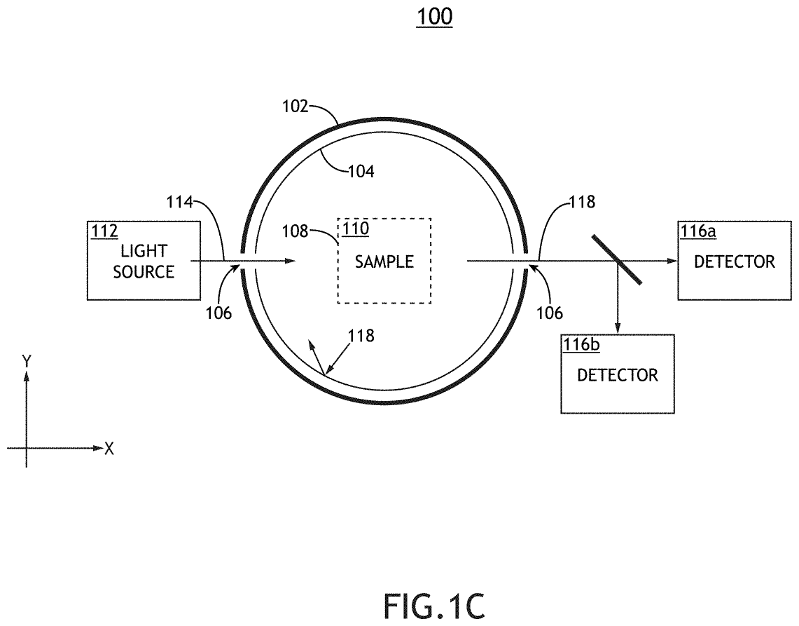

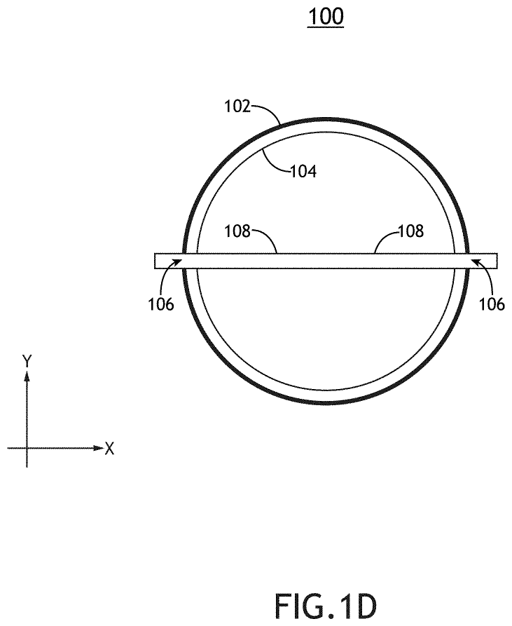

Referring now to FIGS. 1A-10, systems and methods for temporal measurements of dynamic samples are described in greater detail, in accordance with one or more embodiments of the present disclosure.

FIG. 1A is a conceptual diagram of a dynamic scattering measurement system 100, in accordance with one or more embodiments of the present disclosure.

In some embodiments, the dynamic scattering measurement system 100 includes a diffusive cavity 102 including a reflective internal surface 104 and one or more ports 106 for the passage of light into or out of the diffusive cavity 102. In some embodiments, the diffusive cavity 102 is suitable for enclosing a sample chamber 108 for holding a sample 110 (e.g., a sample of a dynamic medium, a complex medium, a disordered medium, or the like). In some embodiments, the dynamic scattering measurement system 100 includes a light source 112 to generate measurement light 114, which may be directed into the diffusive cavity 102 through one of the ports 106 for interaction with the sample 110.

The reflective internal surface 104 of the diffusive cavity 102 may be formed from any suitable material having a high reflectivity such as, but not limited to, silica or barium sulfate. Further it is contemplated herein that the selection of the reflective internal surface 104 may be based in part on the wavelength of the measurement light 114.

The diffusive cavity 102 may have any shape suitable for providing uniform diffuse illumination of the sample 110 such as, but not limited to a sphere. Further, the diffusive cavity 102 may be formed as a cavity within a solid block of material or may be formed from various connected components arranged to form the cavity. In this way, the diffusive cavity 102 may provide uniform illumination of the sample 110 from all directions such that measurement light 114 with all possible wave vectors may interact with the sample 110 during a measurement. Further, the measurement light 114 from the light source 112 may be directly incident on the sample 110 (e.g., through the sample chamber 108) or may be incident on the reflective internal surface 104 of the diffusive cavity 102. In some embodiments, the diffusive cavity 102 is an integrating sphere. In some embodiments, the diffusive cavity 102 is a Cauchy cavity suitable for providing diffuse scattering conditions in which a generalized Cauchy invariant for dynamic media as disclosed herein is valid.

The light source 112 may include any light source known in the art suitable for providing measurement light 114. In some embodiments, the light source 112 is a coherent light source to provide coherent measurement light 114. In some embodiments, the light source 112 is a monochromatic or narrow-band light source to provide monochromatic or narrow-band light. Further, the measurement light 114 may have any wavelength or range of wavelengths suitable for optical scattering measurements. For example, the measurement light 114 may have, but is not limited to, wavelengths in ultraviolet, visible, or infrared spectral regions.

In some embodiments, the dynamic scattering measurement system 100 includes one or more detectors 116 to detect scattered light 118 through at least one of the ports 106. The detectors 116 may include any type of optical detectors known in the art including, but not limited to, photodiodes, avalanche photodiodes, or photomultipliers. In some embodiments, at least one detector 116 a single-photon detector such as, but not limited to, single-photon avalanche photodiodes (SPAD). In some embodiments, at least one detector 116 is a multi-pixel detector. In this way, the detector 116 may provide spatially-resolved images of the sample 110.

The one or more detectors 116 may be arranged in any manner suitable for detecting scattered light 118 from the sample 110.

In some embodiments, the detectors 116 receive light from different ports 106 of the diffusive cavity 102 to capture scattered light 118 at different angles with respect to the sample 110 and/or the incident measurement light 114. FIG. 1B is a conceptual schematic of the dynamic scattering measurement system 100 including two detectors 116a,b distributed to receive light from two ports 106 of a diffusive cavity 102, in accordance with one or more embodiments of the present disclosure. It is contemplated herein that the reflective internal surface 104 of the diffusive cavity 102 may provide a uniform distribution of light throughout the diffusive cavity 102 such that the detectors 116 may receive scattered light 118 from ports 106 located at any angle with respect to the sample 110 and/or the incident measurement light 114. However, in some embodiments, the detectors 116 receive scattered light 118 from ports 106 located away from a direct line of the measurement light 114 from the light source 112.

In some embodiments, the detectors 116 receive light from a single port 106. FIG. 1C is a conceptual schematic of the dynamic scattering measurement system 100 including two detectors 116 distributed to receive light from a single port 106 of a diffusive cavity 102, in accordance with one or more embodiments of the present disclosure.

For example, the dynamic scattering measurement system 100 may include one or more beamsplitters 120 to split light from a particular port 106 to multiple detectors 116.

Referring generally to FIGS. 1B and 1C, any of the detectors 116 may receive light from the same port 106 through which the measurement light 114 enters the diffusive cavity 102 or a different port 106. In this way, the diffusive cavity 102 may have as few as one port 106. However, it is to be understood that the diffusive cavity 102 may generally have any number of ports 106. Further, in some embodiments, one or more ports 106 may be optionally closed or opened to selectively control the losses of the diffusive cavity 102 and/or allow a selected configuration of ports 106 for use in a measurement.

The light source 112 and/or the detectors 116 may further be coupled to the diffusive cavity 102 using any suitable technique. In some embodiments, any of detectors 116 or the light source 112 are coupled to ports 106 of the diffusive cavity 102 through fiber couplers. In this way, light may be coupled into or out of the diffusive cavity 102 using optical fibers (e.g., single-mode fibers, multi-mode fibers, or the like). In some embodiments, any of detectors 116 or the light source 112 are coupled to ports 106 of the diffusive cavity 102 through free-space techniques.

The sample chamber 108 may include any type of vessel of any shape suitable for containing the sample 110. In some embodiments, the sample chamber 108 is formed as a transparent vial, which may be internally mounted to the diffusive cavity 102 using support structures (not shown). In some embodiments, the sample chamber 108 includes an inlet and an outlet that extend through ports 106 in the diffusive cavity 102 to allow for samples 110 to be loaded prior to a measurement, removed after a measurement, and/or flowed during a measurement without opening the diffusive cavity 102. FIG. 1D is a is a conceptual schematic of the dynamic scattering measurement system 100 including a tubular sample chamber 108 extending between ports 106 of the diffusive cavity 102, in accordance with one or more embodiments of the present disclosure. In this configuration, the sample chamber 108 may be fully transparent or may have a transparent portion (e.g., within a central region of the diffusive cavity 102).

In some embodiments, the dynamic scattering measurement system 100 includes a controller 122 communicatively coupled to at least the detectors 116. In some embodiments, the controller 122 includes one or more processors 124 configured to execute program instructions causing the processors 124 to execute various steps disclosed herein. For example, the one or more processors 124 may be configured to execute a set of program instructions maintained in a memory device 126, or memory. In this way, the controller 122 may carry out any steps associated with present disclosure including, but not limited to, receiving data from the detectors 116, analyzing the data, processing the data, or determining one or more measurements of with the sample 110 based on the data. As an illustration, the controller 122 may receive signals from two detectors 116, generate a cross-correlation of the signals g(τ), and determine time-varying properties of the sample 110 such as one or more microscopic characteristic times (τ₀). As another illustration, the controller 122 may receive separate signals from one or more detectors and determine time-varying properties of the sample 110 such as one or more microscopic characteristic times (τ₀) based on the separate signals.

The controller 122 may then determine various additional properties of the sample 110 (or particles therein) based on the time-varying properties such as, but not limited to, information about constituent particle sizes or shapes, concentration, viscosity, or viscoelastic moduli. In some embodiments, the controller 122 measures changes in the time-varying properties of the sample 110 over longer timescales (e.g., timescales longer than the microscopic characteristic time (τ₀)). In this way, the dynamic scattering measurement system 100 may be suitable for detecting and/or monitoring changes of the sample 110 over these longer time-scales. It is contemplated herein that detecting and/or monitoring time-varying changes of the sample 110 may be useful for a wide variety of applications including, but not limited to, monitoring biological reactions, chemical reactions, polymerization processes or aggregation processes.

The one or more processors 124 of a controller 122 may include any processor or processing element known in the art. For the purposes of the present disclosure, the term “processor” or “processing element” may be broadly defined to encompass any device having one or more processing or logic elements (e.g., one or more micro-processor devices, one or more application specific integrated circuit (ASIC) devices, one or more field programmable gate arrays (FPGAs), or one or more digital signal processors (DSPs)). In this sense, the one or more processors 124 may include any device configured to execute algorithms and/or instructions (e.g., program instructions stored in memory). In some embodiments, the one or more processors 124 may be embodied as a desktop computer, a server, a tablet computer, a mobile phone, or any other computer system configured to execute a program configured to operate or operate in conjunction with the dynamic scattering measurement system 100, as described throughout the present disclosure.

The memory device 126 may include any storage medium known in the art suitable for storing program instructions executable by the associated one or more processors 124. For example, the memory device 126 may include a non-transitory memory medium. By way of another example, the memory device 126 may include, but is not limited to, a read-only memory (ROM), a random-access memory (RAM), a magnetic or optical memory device (e.g., disk), a magnetic tape, a solid-state drive and the like. It is further noted that the memory device 126 may be housed in a common controller housing with the one or more processors 124. In some embodiments, the memory device 126 may be located remotely with respect to the physical location of the one or more processors 124 and the controller 122. For instance, the one or more processors 124 of the controller 122 may access a remote memory (e.g., server), accessible through a network (e.g., internet, intranet and the like).

In one embodiment, the dynamic scattering measurement system 100 includes a user interface 128 communicatively coupled to the controller 122. In one embodiment, the user interface 128 may include, but is not limited to, one or more desktops, laptops, tablets, mobile phones, or the like. In another embodiment, the user interface 128 includes a display used to display data of the dynamic scattering measurement system 100 to a user. The display of the user interface 128 may include any display known in the art. For example, the display may include, but is not limited to, a liquid crystal display (LCD), an organic light-emitting diode (OLED) based display, or a CRT display. Those skilled in the art should recognize that any display device capable of integration with a user interface 128 is suitable for implementation in the present disclosure. In another embodiment, the user interface 128 may include a user input device enabling a user to input selections and/or instructions such as, but not limited to, a touchscreen device, a keyboard, a mouse, or a voice-activated input.

Referring generally to FIGS. 1A-1C, in some embodiments, the dynamic scattering measurement system 100 includes additional components to control environmental conditions in the sample chamber 108 or the diffusive cavity 102 such as, but not limited to, temperature, pressure, humidity 20, or atmospheric composition. For example, the dynamic scattering measurement system 100 may include one or more regulators 130 to control the environmental conditions (e.g., temperature regulators, pressure regulators, humidity regulators, atmospheric regulators, or the like). Further, in some embodiments, though not shown, the diffusive cavity 102 is placed within a chamber to facilitate control of the environmental conditions. The dynamic scattering measurement system 100 may additionally include various sensors 132 to monitor the environmental conditions for the purposes of controlling the environmental conditions. As an illustration, the controller 122 may receive data from one or more environmental sensors 132 and use this data for feedback and/or feedforward control of one or more regulators 130.

In some embodiments, dynamic scattering measurement system 100 includes one or more field generators 134 to expose the sample 110 to one or more external fields, which may be static or time-varying. For example, the field generators 134 may include an electric field generator to expose the sample 110 to an electric field. As another example, the field generators 134 may include a magnetic field generator to expose the sample 110 to a magnetic field. As another example, the field generators 134 may include an acoustic field generator to expose the sample 110 to an acoustic field. It is contemplated herein that such applied external fields may influence the measurable properties of the sample 110 and/or their temporal evolution. The dynamic scattering measurement system 100 may thus generate measurements of time-varying properties of the sample 110 with one or more applied external fields, without the one or more applied external fields, or in response to variations of the one or more external fields when characterizing the sample.

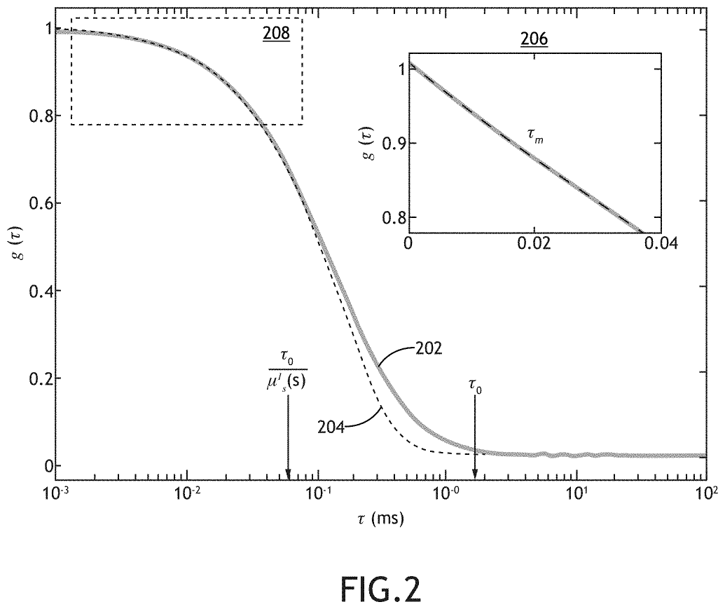

Referring now to FIGS. 2-10, optical scattering regimes and measurements using the dynamic scattering measurement system 100 are described in greater detail, in accordance with one or more embodiments of the present disclosure. It is noted that FIGS. 2-10 generally describe measurements based on two detectors 116 (e.g., cross-correlation measurements between two detectors 116). However, it is to be understood that FIGS. 2-10 and the associated descriptions are provided solely for illustrative purposes and should not be interpreted as limiting. Rather, the dynamic scattering measurement system 100 may provide measurements of time-varying characteristics of a sample 110 using detection signals from one or more detectors 116 of any type including, but not limited to, single-pixel detectors or multi-pixel detectors.

Fluctuations of scattered intensity encode the time-varying properties of complex media. To extract the information, inverse problems must rely on accurate description of the process of light matter interaction. For instance, the dynamic light scattering (DLS) procedure was designed for the strict limit of deterministic single scattering. When the interaction enters a regime of multiple scattering, the dynamic inverse problem can be approached using a diffusing wave spectroscopy (DWS) methodology. In this case, however, the constraint is that one requires additional information about the path-length (s) of light through the medium. What is needed, practically, is the probability density function P(s) of optical path-lengths inside the medium, a quantity that intimately depends on the experimental circumstances. Because analytical solutions for P(s) exist only for certain special cases, the approach has a limited applicability.

Certain invariance properties exist for extreme conditions of interaction between monochromatic light and finite size inhomogeneous media. For instance according to Cauchy's mean-chord-theorem, in the ballistic regime, the mean path length [symbol] s [symbol] =∫₀^{∞}P(s)ds of the scattered light is solely determined by the volume and the effective surface of the medium. A similar conclusion can be reached for a diffusive regime: [symbol] s [symbol] depends on the size of the medium but not on the microscopic characteristics of the interaction process. Importantly, these conclusions are valid only when both the illumination and detection are homogeneous and isotropic across the surface.

It is contemplated that the systems and methods disclosed herein utilize a generalization of this essential property to dynamic regions of interaction. In particular, this fundamental invariance permits extracting the time-scale of medium's dynamics irrespective of the specific experimental geometry and the scattering regime such that time-varying properties can be obtained without detailed knowledge of P(s).

Considering light scattering from a complex system of identical particles, which diffuse thermally with a Stokes-Einstein diffusion coefficient (D), a straightforward extension can account for biased diffusion as well as the many body collective effects. For an incident field with wavenumber k₀, the fluctuations of light have a microscopic characteristic time τ₀=1/(2Dk₀²). The goal of a generic experiment is to retrieve τ₀ from the measured field-field correlation function

$$g ( τ ) = \frac{{\vert 〈 E ( 0 ) {E}^{*} ( τ ) 〉 \vert}^{2}}{{〈 \vert E ( 0 ) \vert 〉}^{2}} ,$$

which is characterized by a measured correlation time τₘ. The challenge is that these two time scales are not necessarily the same. Their ratio τ₀/τₘ depends on the strength of light matter interaction as quantified by the reduced scattering coefficient μₛ′ that defines different scattering regimes, as suggested in FIG. 2.

FIG. 2 is a plot of measured field-field correlation function in the strong scattering regime of light-matter interaction, in accordance with one or more embodiments of the present disclosure. As illustrated in FIG. 2, the field-field correlation function g(τ) (solid line 202) in the strong scattering regime of light-matter interaction depends on both τ₀ and the pathlength distribution P(s). In the short time limit,

$$τ ≪ \frac{{τ}_{0}}{{μ}_{s}^{′} 〈 s 〉} ,$$

the perceived time-scale τₘ is found from the single exponential decay of g(τ) (black dashed line 204). Thus, τ₀ can be extracted using solely the mean [symbol] s [symbol] without having to know the explicit form of P(s) or its higher-order moments. Further, the inset plot 206 depicts these values for a portion of the selected region 208.

When μₛ′ is much smaller than the size of the medium, the perceived characteristic time becomes τₘ=1/(Dq²), where q=2k₀ sin (θ/2) is the corresponding transfer wave-vector as defined by the specific experimental geometry. In this single scattering regime, the macroscopic size and shape of the medium are irrelevant, and one can easily find that τ₀/τₘ=2 sin²(θ/2). This regime is denoted by the black dashed line 204 in FIG. 2, where τₘ/τ₀ is simply a geometry-dependent constant.

With increasing μₛ′, the interactions enter a regime of stronger light-matter coupling where the relation between τₘ and τ₀ becomes rather complicated. Experimentally, due to the internal dynamics of the medium, random phases will accumulate at the detector and contribute to the measured field-field correlation. It has been shown that the coupling between the intrinsic dynamics τ₀ and the measured field-field correlation is determined by the distribution of photon path lengths P(s). When τ<<τ₀, the measured field-field correlation function turns out to be:

$$g ( τ ) = {\int }_{0}^{∞} P ( s ) exp ( - \frac{s {μ}_{s}^{′} τ}{{τ}_{0}} ) ds . \qquad ( 1 )$$

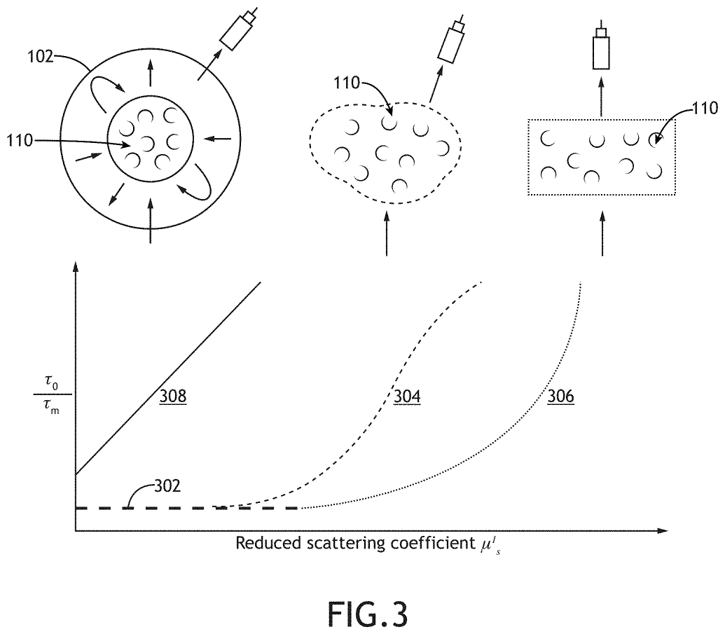

In general, a series of higher-order moments of P(s) are necessary to describe g(τ) in an intermediate regime of interaction as shown in FIG. 3.

FIG. 3 is a conceptual plot of a log-log representation of ratio between the microscopic τ₀ and perceived τₘ correlation times for different regimes of light-matter interaction, in accordance with one or more embodiments of the present disclosure. In the single scattering limit, τ₀/τₘ is purely a geometry constant (black dotted line 302). When the coupling increases, τ₀/τₘ depends strongly on μₛ′, the measurement geometry, and also the macroscopic properties of the medium, as suggested by the middle line 304. Analytical solutions may be found only for limiting situations such as, for example, transmission through a two-dimensional diffusive and infinitely extended slab (right line 306). The left line 308 depicts the circumstances associated with a diffusive cavity 102 as disclosed herein.

However, in the limit τ<<τ₀/(μₛ′ [symbol] s [symbol] ), the measured correlation time τₘ depends only on the first moment [symbol] s [symbol] of P(s), and, consequently, τ₀/τₘ=μₛ′ [symbol] s [symbol] . This means that, in the limit of very short times, the outcome of the dynamic measurement is solely determined by [symbol] s [symbol] and knowledge of the entire P(s) is not necessary.

For example, following the diffusive wave spectroscopy theory, in multiple scattering regime, the field-field correlation function g(τ) is given as follows:

$$g ( τ ) = {\int }_{0}^{∞} P ( s ) exp ( - \frac{\frac{s}{{l}^{*}} τ}{{τ}_{0}} ) ds . \qquad ( 2 )$$

As described previously herein, P(s) is the probability distribution of photon path length, while τ₀=1/(2k²D) is the time donating the dynamics of a single object. Note that this equation works for the regime where τ<<τ₀. A typical routine to retrieve τ₀ is to perform fitting of g(τ) within this range using the knowledge of P(s). Using this technique, detailed knowledge of P(s) is thus necessary.

However, the same goal can also be achieved by using only the short time limit where

$$τ ≪ \frac{{l}^{*}}{〈 s 〉} \cdot {τ}_{0} .$$

In this case, one can take the Taylor expansion of the correlation function and obtain:

$$\begin{matrix}g ( τ ) \approx {\int }_{0}^{∞} P ( s ) [ 1 - \frac{s}{{l}^{*}} \cdot \frac{τ}{{τ}_{0}} ] ds \\ = {\int }_{0}^{∞} P ( s ) ds - \frac{1}{{l}^{*}} \cdot \frac{τ}{{τ}_{0}} {\int }_{0}^{∞} P ( s ) ds \\ = 1 - \frac{1}{{l}^{*}} \cdot \frac{τ}{{τ}_{0}} \cdot 〈 s 〉 \approx exp ( - \frac{〈 s 〉}{{l}^{*}} \cdot \frac{τ}{{τ}_{0}} ) .\end{matrix} \qquad ( 3 )$$

Eq. (3) suggests that, in this limit, the field-field correlation function can be approximated by a single exponential. Its characteristic time is only associated with the first moment of the optical path-length [symbol] s [symbol] , instead of the detailed P(s). Therefore, one can define the characteristic time τₘ of the g(τ) at the short time limit as

$${τ}_{m} = \frac{{l}^{*}}{〈 s 〉} \cdot {τ}_{0} . \qquad ( 4 )$$

In general, both [symbol] s [symbol] and P(s) depend on (i) the microscopic characteristics of scattering events, (ii) the macroscopic properties of medium, and (iii) the measurement configuration. This is why analytical solutions can only be found in certain limiting situations such as, for instance, of diffusive transmission through a slab, which is denoted by the right line 306 in FIG. 3. We note that, even in this case, the solution accuracy strongly depends on the applicability of the diffusion approximation as well as the detailed boundary conditions. For other geometries as suggested by the middle line 304 in FIG. 3, numerical approaches are typically necessary. Alternatively, one could directly measure P(s) by time-of-flight or broadband interferometric techniques, but this process will still be geometry dependent. For at least these reasons, it remains a challenge to determine the ratio τ/τₘ generally and, therefore, to extract the microscopic dynamic characteristic time τ₀ in a general manner irrespective of the macroscopic properties of the medium.

One way to overcome the requirement of detailed knowledge of P(s). The first is to try to enforce the single scattering regime, but this is often difficult to ensure in practice.

Referring again to FIGS. 1A-1C, it is contemplated herein that another approach is to transfer the light-matter interaction into a fully-developed multiple scattering regime such that [symbol] s [symbol] may be easily determined. It is further contemplated herein that this may be achieved by appealing to the so-called generalized Cauchy invariance property.

When radiation interacts diffusively with a bounded medium (e.g., a sample 110), the mean path length of light (e.g., measurement light 114) is simply determined by the overall volume V and surface area Σ of the medium through which it propagates such that

[symbol]

s [symbol] =4·V/Σ.

It is noted that Eq. (5) holds when the illumination and detection is performed uniformly across the medium. Practically, light must be injected and detected homogeneously with all possible wave-vectors.

It is further contemplated herein that Eq. (5) and the associated assumptions holds when a sample 110 is enclosed in a diffusive cavity 102 as depicted in FIGS. 1A-1C. In this configuration, the diffusive cavity 102 homogenizes the field of the measurement light 114 across a surface of the sample 110 (e.g., as defined by the sample chamber 108) and thus ensures that the conditions for Cauchy invariance disclosed herein are satisfied. In addition, the reflective internal surface 104 of the diffusive cavity 102 increases dramatically the average residence time of photons of the measurement light 114 within the sample 110, which, in turn, ensures a diffusive regime of light-matter interaction. Under these conditions, it can be shown that:

$$\frac{{τ}_{0}}{{τ}_{m}} \cdot \frac{1}{{μ}_{s}^{′}} = 4 \cdot \frac{V}{Σ} , \qquad ( 6 )$$

which constitutes a generalization of the Cauchy invariant property to dynamic media (e.g., a sample 110). The meaning of Eq. (6) is that, for light diffusing in a diffusive cavity 102 (e.g., a Cauchy cavity), the product between the averaged number of dynamic scattering events and the strength of light matter interaction depends in part or in full on the macroscopic properties of the sample 110.

In practice, the volume and surface are effective macroscopic parameters associated both the physical dimensions of the sample 110 and the optical properties of the diffusive cavity 102. As detailed below, the refractive index contrast n at the physical boundary of the sample 110 (e.g., as defined by the sample chamber 108) and the possible cavity loses η can be accounted for in a practical formulation for the averaged mean path:

$$〈 s 〉 = 4 \cdot \frac{{V}_{eff}}{{Σ}_{eff}} = 4 \cdot \frac{V {n}^{2}}{Σ η} . \qquad ( 7 )$$

Here, n² is a refractive-index correction for the radiative transfer equation solution in a medium subjected to the aforementioned isotropic and homogeneous radiation condition, while η extends the properties of the diffusive walks in the bounded domains.

It is contemplated herein that Eq. (7) may thus apply to the conditions provided by a diffusive cavity 102 as disclosed herein (e.g., as illustrated in FIGS. 1A-1C).

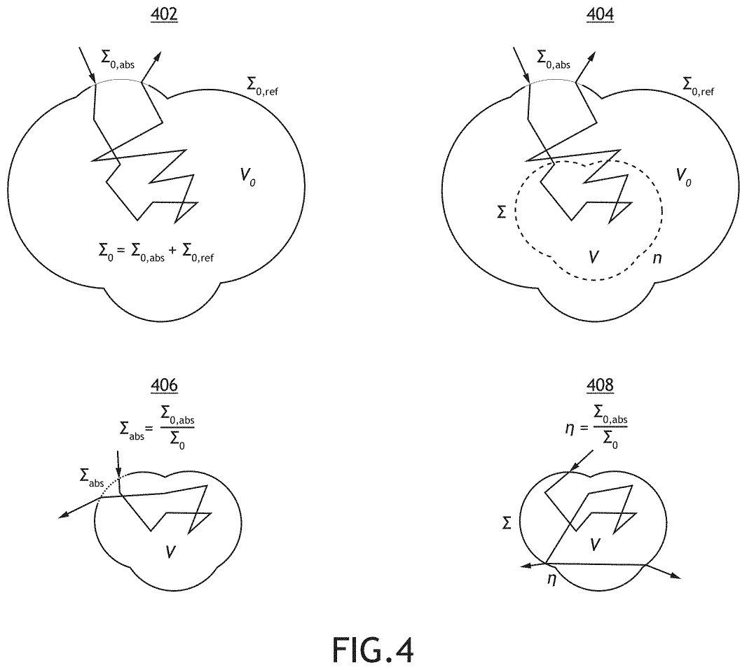

Referring now to FIG. 4, invariance properties of diffusive light in bounded domains associated with Eq. (7) are described in greater detail, in accordance with one or more embodiments of the present disclosure. In particular, an invariance property for diffusive light in a bounded domain with boundary conditions (e.g., Eq. (7)). It is noted that such an invariance property has been rigorously derived only for diffusive walks in bounded domains and taking into account the boundary condition as well as the subdomain. For diffusive light, the boundary conditions with refractive index mismatch has been examined before. The invariance property of diffusive walks to diffusive light in bounded domains is extended herein and associated with the temporal measurements inside a diffusive cavity 102.

FIG. 4 includes conceptual schematics of diffusive walks under various conditions, in accordance with one or more embodiments of the present disclosure. In particular, panel 402 illustrates diffusive walks in bounded domains with absorbing Σ_{0,abs} and reflective Σ_{0,ref} boundaries; panel 404 illustrates diffusive walks in the subdomain of a bounded system; panel 406 illustrates the equivalent system in panel 404, but with an absorbing boundary Σ_{abs} and a corresponding reflective boundary; and panel 408 illustrates a similar system of panel 406, but with uniform partial reflective boundary (η represents the loss of the boundary).

To start with, consider the diffusion walks in three dimensions inside a bounded complex system of volume V₀ as depicted in panel 402 of FIG. 4. The outer envelope of a bounded system Σ₀ can be considered as the sum of an absorbing component with surface Σ_{0,abs} and one reflective with surface Σ_{0,ref} (with unity reflectivity) to practically have Σ₀=Σ_{0,abs}+Σ_{0,ref}. As this is a purely diffusive system, any diffusive walk would eventually reach the absorbing portion of the boundary. It can be assumed that the diffusive walks start only on the surface of the absorbing boundary, with isotropic and uniform intensity. It has been shown that, under such conditions, the mean path length of these diffusive walks is:

$${〈 s 〉}_{{Σ}_{0 , a b s}} = \frac{4 {V}_{0}}{{Σ}_{0 , a b s}} . \qquad ( 8 )$$

Next, one can consider a subdomain of volume V inside V₀, as illustrated in panel 404 of FIG. 4. In this case, if one considers the random walks that start and end on the absorbing boundary, the mean path length of the diffusive walks inside the subdomain V are:

$${〈 s 〉}_{{Σ}_{0 , a b s}} = \frac{4 V}{{Σ}_{0 , abs}} . \qquad ( 9 )$$

Now, to extend this conclusion to diffusive light inside a bounded system, a correction regarding the volume must be made to account for the mismatch to the isotropic and homogeneous radiation. This is due to the refractive index mismatch of the layer between the subdomain V and other regime of V₀, which can be characterized by the refractive index contrast n. The n² term comes from the correction over the irradiance density between the mismatch layer, which is a consequence of the stationary solution of the radiative transfer equation (RTE). Thus, one can write

$${〈 s 〉}_{{Σ}_{0 , a b s}} = \frac{4 V {n}^{2}}{{Σ}_{0 , abs}} . \qquad ( 10 )$$

It is noted herein that, Eq. (10) describes the mean path length of photons inside V whose trajectories start and end in the absorbing boundary Σ_{0,abs}. However, it is contemplated herein that under experimental conditions, only diffusive walks inside the volume V will contribute to the measured temporal correlation function.

This can be approximated by decreasing the size of the bounded domain until V≈V₀ and Σ≈Σ₀, as shown in FIG. 4(c). This will lead to

$${〈 s 〉}_{{Σ}_{a b s}} = \frac{4 V {n}^{2}}{{Σ}_{a b s}} , \\ where {Σ}_{a b s} = Σ \times {Σ}_{0 , a b s} / {Σ}_{0} . \qquad ( 11 )$$

Finally, one can define the loss at the boundary η=Σ_{0,abs}/Σ₀ by assuming that the loss appears uniformly across the surface of the subdomain. This means that instead of applying an absorbing and reflected boundary, it is equivalent to consider a system with boundaries of uniform but non-unity reflectivity (R=1−η). It is noted that this will not affect the launching conditions for the diffusive random walk. The corresponding physical picture is shown in FIG. 4(d). It is also noted that this can be considered as an ergodic-type generalization of the previous invariance property in bounded domains. Therefore, it is straightforward to start from Eq. (11) and obtain the relation for the mean path length of diffusive light in such a system shown above as Eq. (7).

It is contemplated herein that Eq. (7) is obtained in disordered media (e.g., a sample 110), but can be generalized to other dimensionalities by simply replacing the number 4 with dimension-dependent constant η_{d} to provide a more generalized expression:

$${〈 s 〉}_{{Σ}_{a b s}} = {η}_{d} \cdot \frac{V {n}^{2}}{Σ η} , \\ where {η}_{d} = \sqrt{π} ( d - 1 ) Γ [ \frac{d - 1}{2} ] / Γ ( \frac{d}{2} ) \qquad ( 12 )$$

and d represents the dimensionality of the system.

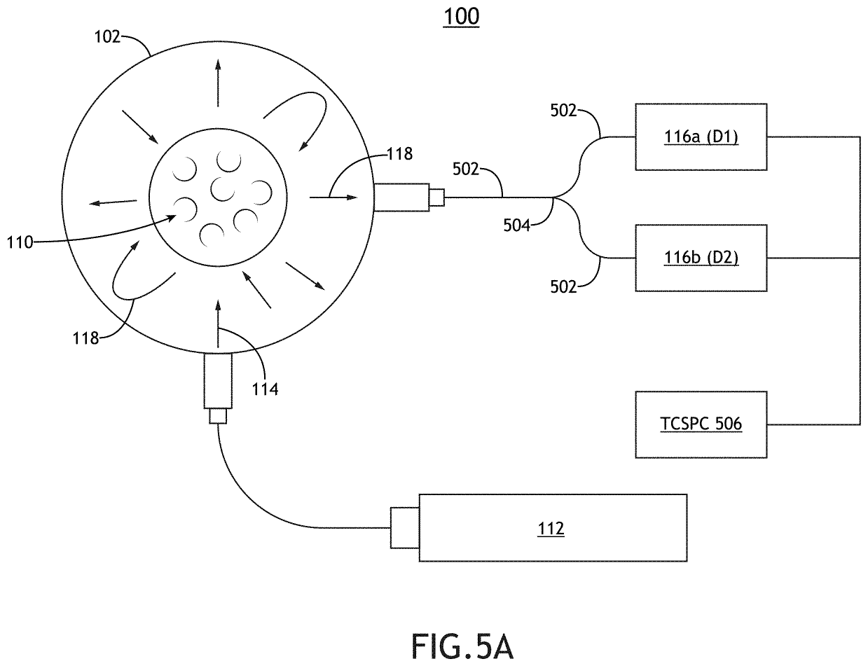

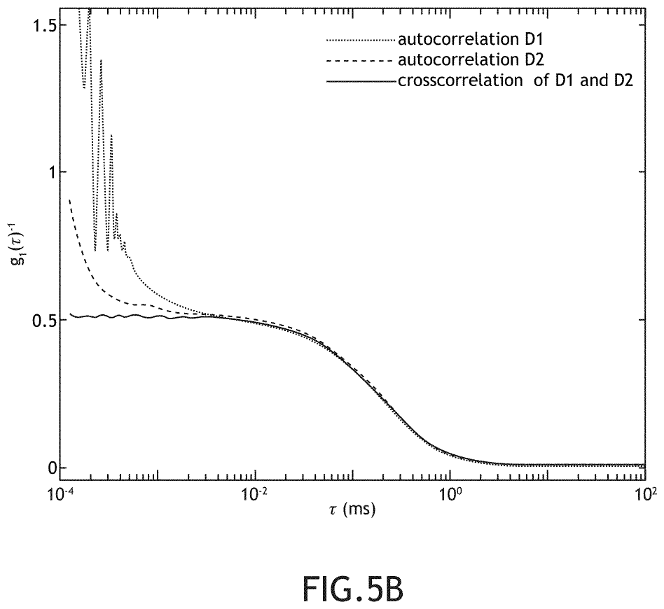

Referring now to FIGS. 5A-10, experimental demonstrations and validations of non-limiting configurations of the dynamic scattering measurement system 100 are described in greater detail, in accordance with one or more embodiments of the present disclosure.

FIG. 5A is a conceptual view of the dynamic scattering measurement system 100 used for the generation of the data in FIGS. 5B-10, in accordance with one or more embodiments of the present disclosure.

In FIGS. 5A-10, the light source 112 is a green continuous wave laser providing continuous-wave measurement light 114 having a wavelength of 532 nm and a power of 100 mW. The measurement light 114 is coupled into a single mode fiber and directed into a diffusive cavity 102 formed as an integrating sphere with a diameter of approximately 5 cm and having a reflective internal surface 104 with approximately 99% reflectivity. Two single-photon detectors 116 (SPADs) are coupled to a single port 106 of the diffusive cavity 102 through optical fibers 502 and a fiber coupler 504.

The dynamic scattering measurement system 100 further includes a Time-Correlated Single Photon Counting (TCSPC) correlator card 506 is used for measuring the temporal correlation function g(τ) of signals from the two detectors 116, which has the dynamic range from 10⁻⁷ to 10⁻¹ seconds.

FIG. 5B is a plot of the temporal correlation function g(τ) of signals from the two detectors 116 coupled to the diffusive cavity 102 of FIG. 5A, in accordance with one or more embodiments of the present disclosure. It is noted that the after-pulsing of such detectors 116 can cause issues when resolving the g(τ) at very short times, which typically happens around 10⁻⁵ seconds. This effect, however, can be mitigated using the cross-correlation scheme of the measured signals from two independent detectors 116, as shown in FIG. 5B. The total measurement time for each temporal correlation function is 5 minutes in these experiments.

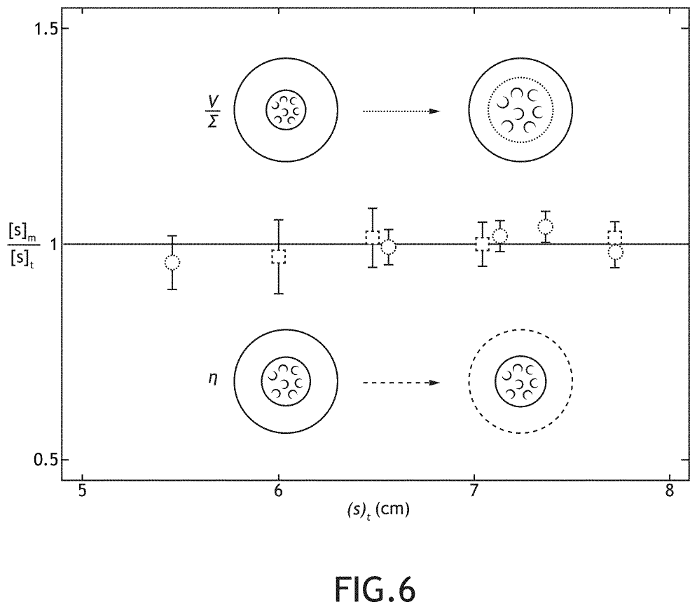

It is contemplated herein that there are several types of losses in the dynamic scattering measurement system 100 as depicted in FIG. 5A including, but not limited to, the non-unity reflection of the reflective internal surface 104, the colloidal absorption, the absorption of the sample 110 generally, or absorption of the sample chamber 108. In the particular non-limiting set of experiments herein, the absorption of the reflective internal surface 104 (R=0.99) and that of the colloidal suspension are negligible. Rather, loss in the dynamic scattering measurement system 100 is primarily due to the absorption of the sample chamber 108. The total loss factor of the dynamic scattering measurement system 100 was measured to be η=0.21 by comparing the configuration depicted in FIG. 5A to a configuration with an empty sample chamber 108 and a reflective port plug, each during a total measurement duration of 1 hour. As shown in FIG. 6, an additional loss factor can be induced by opening more ports on the integrating sphere. Each opened port 106 adds approximately 0.015 loss in this particular case by calculating the ratio between its area and the internal surface of the diffusive cavity 102.

For each measured g(τ), the following process is used to retrieve the characteristic time of the dynamics τ_{c}. First, each g(τ) measurement is normalized from 0 to 1. Next, only g(τ)∈[0.8, 1] is selected to perform a single exponential fitting, in which the decay rate of the exponential function is τₘ. This ensures the condition τ<<l*/( [symbol] s [symbol] τ₀) as discussed previously herein.

FIG. 6 is a plot of a comparison between measured mean path [symbol] s [symbol] ₘ and the prediction [symbol] s [symbol] ₜ from Eq. (7) for dynamic media (e.g., samples 110) with similar microscopic but different macroscopic properties (V/Σ: circles, η: squares), in accordance with one or more embodiments of the present disclosure. The error bars are evaluated by propagating the fitting errors from measurements of g(τ) and indicate 95% statistical confidence.

Eq. (7) may be validated using dynamic media (e.g., samples 110) with different macroscopic properties. The media are suspensions of particles with diameter of 1 μm and volume concentration of 0.1%, which ensures that both the intrinsic time scale τ₀ and the strength of light matter interaction μₛ′ are the same for all samples 110. The results summarized in FIG. 6 correspond to situations where the size of the sample 110 changes (circles) or where the effective cavity surface (e.g., the reflective internal surface 104) is modified (squares) by opening additional ports 106 in the diffusive cavity 102. The experimental value of the mean free path [symbol] s [symbol] ₘ=τ₀/(μₛ′τₘ) is compared to [symbol] s [symbol] ₜ estimated from Eq. (7) by adjusting either the geometry quantity V/I (circles) or the boundary conditions by tuning q (squares). The q is tuned by opening the number of ports 106 of the integrating sphere. As can be seen, the modified Cauchy invariance of Eq. (4) describes very well the experimental data across the entire range of macroscopic parameters.

Two more observations are noted herein. First, the experiment summarized in FIG. 6 provides the first direct experimental demonstration of the invariance property of optical paths in diffusive systems with adjustable reflective and absorbing boundaries. Second, it demonstrates that one can tune the macroscopic parameters of the dynamic scattering measurement system 100 (V, Σ, and q) in a deterministic way to ensure a strong coupling between light and matter in a dynamic sample 110, μₛ′ [symbol] s [symbol] >>1, and therefore create the circumstances where this invariance property can be applied. This means that in a diffusive cavity 102 (e.g., a Cauchy cavity), it is possible to achieve a regime of diffusive interaction for arbitrarily complex media.

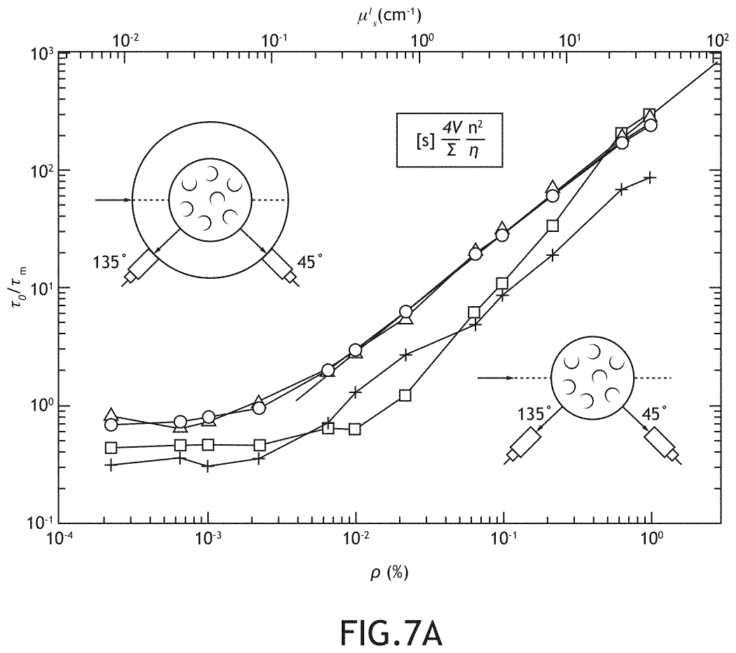

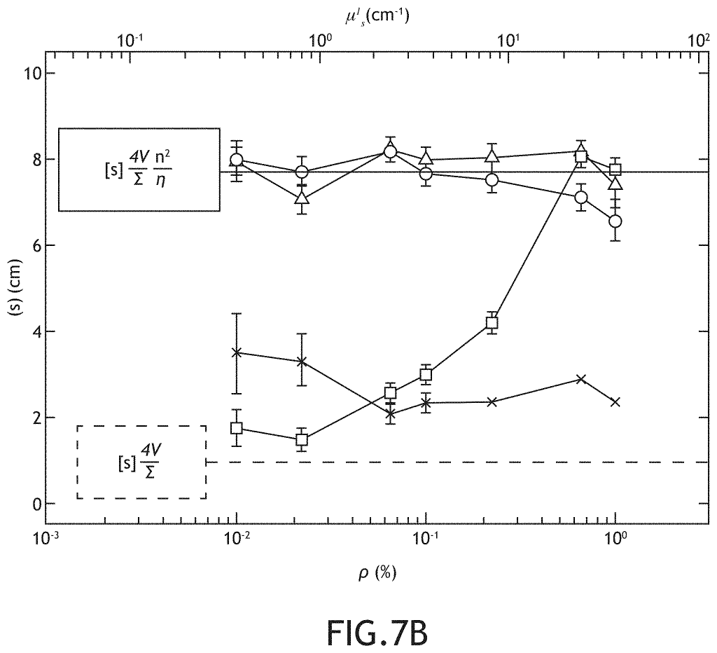

Referring now to FIGS. 7A and 7B, a second series of tests is described. The second series of tests involved weakly scattering media consisting of polystyrene particles with 1 μm diameter and volume fractions p ranging from 0.00022% to 1%. The samples were placed in identical transparent containers and four different experiments were performed for each of them both inside and outside the Cauchy cavity. The experimental geometries and a summary of results are shown in FIGS. 7A and 7B. The conditions of this measurement are such that V/Σ=2.3 mm, n=1.33, and η≈0.21. The intrinsic dynamic time τ₀ is calculated from the Stokes-Einstein equation while μₛ′ is obtained from the corresponding Mie calculations.

In particular, FIG. 7A includes a plot of the ratio τ₀/τₘ for different experimental geometries: inside the disordered cavity at 45° (circles) and 135° (triangles), outside the cavity at 45° (squares) and 135° (plusses), respectively, in accordance with one or more embodiments of the present disclosure. Samples are 1 μm polystyrene spheres in diameter, of different concentration ρ (x axis at the bottom) and μₛ′ (x axis on top). The grey line indicates the prediction of τ₀/τₘ=μₛ′ [symbol] s [symbol] with [symbol] s [symbol] from Eq. (7).

As can be seen in FIG. 7A, the results of the measurements performed outside a diffusive cavity 102 depend significantly on the experimental conditions as expected. In practice, this complicates significantly the procedure of inferring τ₀ for a given μₛ′ as suggested already in FIG. 1. On the other hand, for measurements taken inside the diffusive cavity 102, a clear −1 slope can be seen in the log-log representation for samples with ρ>0.01%. Moreover, this happens irrespective of the angle at which the measurement is performed. It is evident that, in this case, the mean path of diffusive photons [symbol] s [symbol] does not depend neither on the microscopic scattering process nor on the [symbol] specific measurement geometry. It is also noted that the higher absolute values of the effective path-length are higher for measurements inside the diffusive cavity 102, which confirms that inside the cavity light is considerably more diffusive. For samples 110 with ρ<<0.01%, the measurements are deviated from the prediction of Eq. (7), due to the finite photon lifetime within the system.

FIG. 7B includes a plot of the values of mean optical path-length of light, in accordance with one or more embodiments of the present disclosure. Note the strong dependence of outside cavity measurements (squares and pluses, bottom) on both experimental geometry and scattering regime. On the other hand, the values of [symbol] s [symbol] measured inside the cavity (circles and triangles, top) are independent of concentration and are very well described by Eq. (7). The error bars indicate 95% statistical confidence.

In FIG. 7B, the average pathlength is plotted for samples 110 with ρ>0.01%. One can clearly see that [symbol] s [symbol] , remains invariant with respect to the concentration of particles for measurements taken inside the cavity, while it varies considerably other experimental conditions. Most importantly, the value of [symbol] s [symbol] corresponding to measurements inside the diffusive cavity 102 are very well described by the prediction in Eq. (7). Note also the accuracy of the index contrast and cavity loss corrections described in Eq. (7) and indicated by the gray dashed line in FIG. 7B.

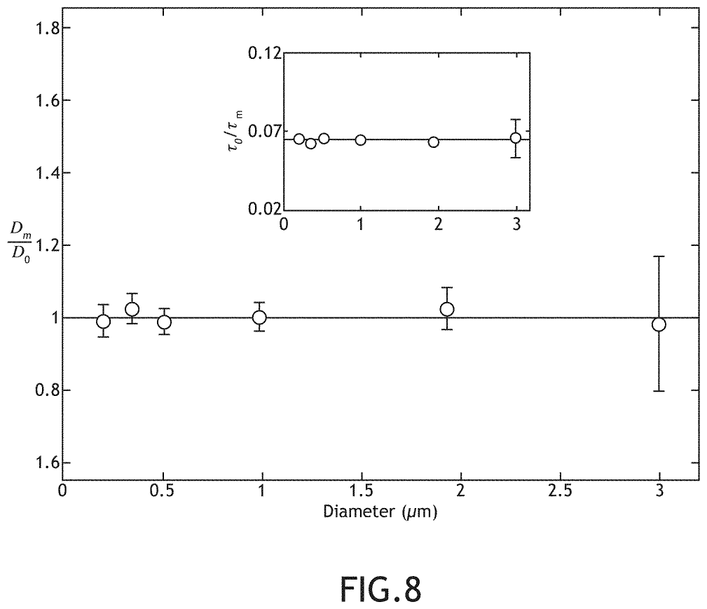

It is further contemplated herein that the dynamic scattering measurement system 100 including a diffusive cavity 102 can be used to retrieve the characteristic time of arbitrarily complex dynamic sample 110. As an illustration, one can adjust the dynamic time by tuning the particle size in the sample 110 while keeping the same μₛ′ of all the samples 110. FIG. 8 is a plot of the diffusion coefficient of colloidal media measured inside the diffusive cavity 102, in accordance with one or more embodiments of the present disclosure. The inset includes the ratio between the measured τₘ and the intrinsic time τ₀ for the sample 110. The error bars indicate 95% statistical confidence. It is noted that the measured diffusion coefficient Dₘ is compared with the value D₀ predicted by the Stokes-Einstein equation and the results are summarized in FIG. 8. The relatively larger error for the 3 μm particles is due to significant hydrodynamic interactions in this case. As evident, the expected internal dynamics is recovered very well in all cases even without knowing the actual P(s).

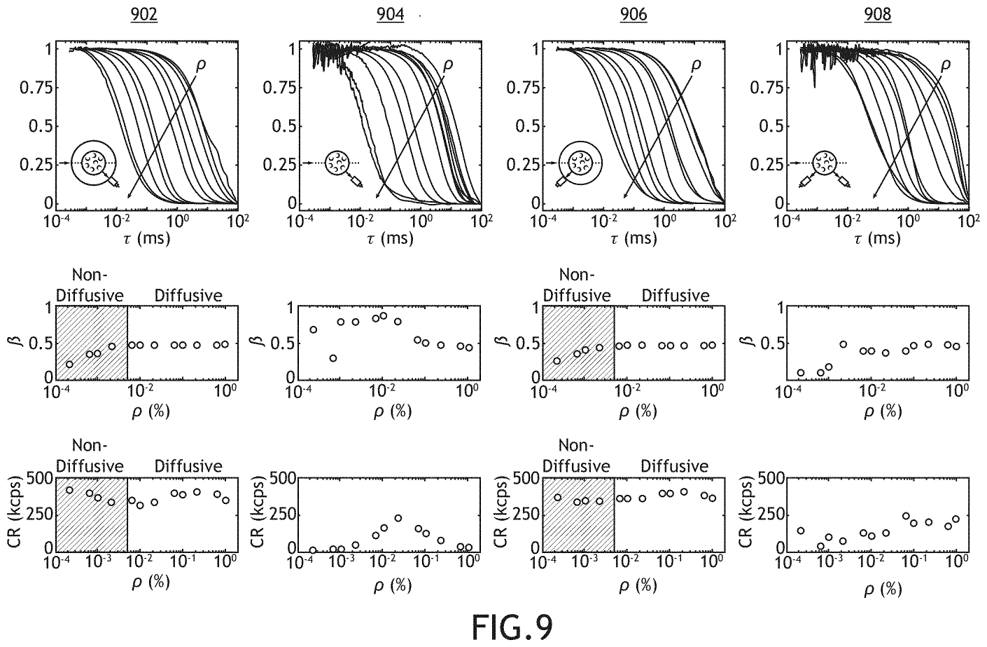

Referring now to FIG. 9, an additional comparison of measurements taken with and without a diffusive cavity 102 are described in greater detail, in accordance with one or more embodiments of the present disclosure.

FIG. 9 includes various plots providing a comparison of measurements taken at different geometries over different range of concentrations ρ, in accordance with one or more embodiments of the present disclosure. In particular, FIG. 9 illustrates measurements taken inside (panel 902) and outside (panel 904) the diffusive cavity 102 (here an integrating sphere) at 45°, along with measurements taken inside (panel 906) and outside (panel 908) the diffusive cavity 102 at 135°. In each panel, the first column includes a plot of the normalized field-field correlation function g(τ) (the arrow indicates the increasing of concentrations ρ), the second column includes a plot of β retrieved from the Siegert relation, and the third column includes a plot of count rates measured by both detectors 116.

The experiments illustrated in FIG. 9 utilized dynamic samples 110 including polystyrene particles of 1 μm diameter in water, with different volume concentrations ρ, ranging from 0.00022% to 1%. The sample chamber 108 in each case was a transparent cylindrical bottle with dimensions of approximately 9 mm in diameter and approximately 15 mm in height). It is noted that the size of the samples 110 were selected to be substantially smaller than the diameter of the diffusive cavity 102 to ensure the homogeneous illumination condition. The sample chamber 108 was placed at the center of the diffusive cavity 102 and fixed to a port plug. Each sample 110 was then placed inside the integrating sphere for 3 minutes before taking the measurement. Measurements inside and outside the diffusive cavity 102 were taken for the same samples 110 for comparison. The duration of each measurement was 5 minutes. The illumination power of the measurement light 114 was approximately 20 mW. To provide a fair comparison, measurements taken outside the diffusive cavity 102 were taken with a source-sample distance and sample-detector distance of 2.5 cm to match the radius of the diffusive cavity 102.

For each measurement, three independent parameters were collected: the correlation function g(τ), the contrast of the correlation function β, and the count rate of the detectors. The values of g(τ) and β are connected through the Siegert relation g_{I}(τ)=1+βg(τ), where g_{I}(τ) represents the intensity-intensity correlation function. Typically, β strongly depends on the experimental geometry. In this experiment, it represents the effect of multiple speckle integration and two polarization states and might be used as an indicator to determine whether diffusion approximation is valid. Experimentally, β can be obtained from β=g_{I}(τ→0).

The corresponding characteristic time of the dynamics for each g(τ) are shown in FIG. 7A. Several observations are noted herein. First, due to the high reflectivity of the diffusive cavity 102, the measurements taken inside the diffusive cavity 102 have higher count rates compared to outside measurements. Otherwise, the count rates remains unchanges when ρ changes, indicating that the systems and methods disclosed herein are robust and do not require any adjustments for a specific measurement. Also, the contrast β is constant (˜0.48) for both cases in the diffusion regime (large φ. As illustrated in FIG. 8, the diffusive cavity 102 dramatically decreased (by more than one order of magnitude) the concentration limit for which the diffusion approximation can be applied. We note that the value of β is not unity mainly due to the use of nonpolarizing single mode fibers that support two polarized modes. Finally, the measurements taken inside the cavity are not sensitive to the angle of detection, while the measurements taken outside the cavity vary substantially in an unpredictable manner when the angle of detection changes. This phenomenon is observed for all three descriptors, g(τ), β and the count rate.

Referring now generally to FIGS. 1A-9, it is contemplated herein that the measurements of the time-varying properties of dynamic samples 110 (e.g., a characteristic time τ (e.g., a microscopic characteristic time), the cross-correlation g(τ), or the like) may form the basis of a wide range of additional measurements. In this way, the systems and methods disclosed herein may be used to provide a wide range of measurements suitable for a wide range of applications. For example, measurements of the temporal dynamics of a complex sample 110 may provide the basis for measurements of particle size, shape, or viscosity of the sample 110.

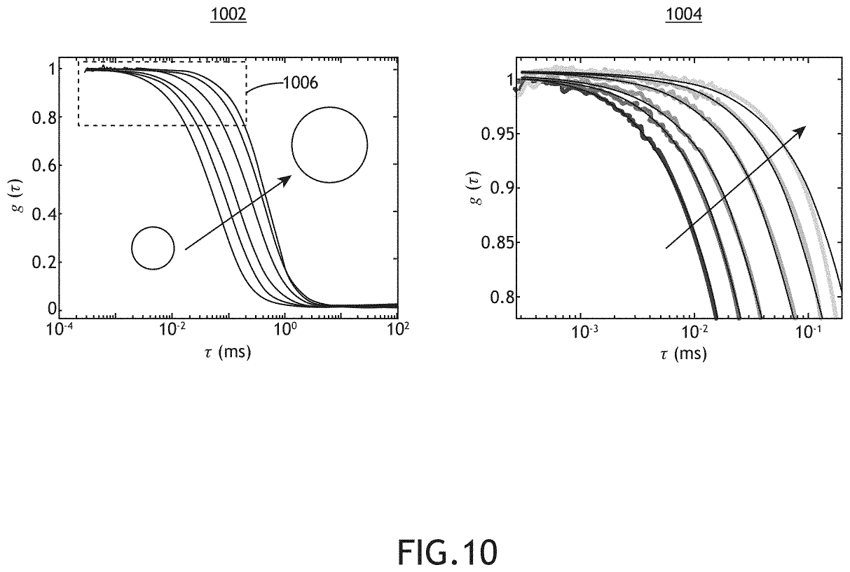

As a an illustration, FIG. 10 includes a plot of experimentally-measured cross-correlation g(τ) for samples 110 having varying particle sizes (e.g., increasing with the arrow in the figure), in accordance with one or more embodiments of the present disclosure. In particular, panel 1002 includes a plot of g(τ) generated using the systems and methods disclosed herein, while panel 1004 includes an expanded view of the region 1006 including experimental fits of the data in panel 1002 using single exponential functions as disclosed herein. For the experiments in FIG. 10, the samples 110 included polystyrene particles of different diameter (from 200 nm to 3 μm) in a transparent cylindrical sample chamber 108 with known volume V and area Σ. The loss of the dynamic scattering measurement system 100 was pre-determined by calibration measurements. Therefore, the mean path of photons [symbol] s [symbol] was calculated theoretically using Eq. (7). The l* of these samples was adjusted to be the same by varying the concentration according to Mie calculations, which is 5 mm. The experimental data with corresponding fitting are shown in FIG. 10. The retrieved τₘ using Eq. (5) as well as D₀ is shown in FIG. 8.

As illustrated in FIG. 10, measurements of g(τ) using the systems and methods disclosed herein clearly illustrate a variation in g(τ) as a function of particle size. In this way, the systems and methods disclosed herein may be adapted to provide particle size measurements based on the temporal dynamics.

Referring now to FIG. 11, FIG. 11 is a flow diagram illustrating steps performed in a method 1100 for measuring time-varying properties of a dynamic medium, in accordance with one or more embodiments of the present disclosure. Applicant notes that the embodiments and enabling technologies described previously herein in the context of the dynamic scattering measurement system 100 should be interpreted to extend to the method 1100. It is further noted, however, that the method 1100 is not limited to the architecture of the dynamic scattering measurement system 100.

In some embodiments, the method 1100 includes a step 1102 of directing measurement light 114 (e.g., coherent or partially coherent light) into a diffusive cavity 102 through one of one or more ports 106, where the diffusive cavity 102 includes a reflective internal surface 104 and provides uniform illumination of a sample 110 in the diffusive cavity 102 through diffusive reflection of at least one of the measurement light 114 or scattered measurement light 114 from the sample 110. The diffusive cavity 102 may generally have any shape, size, or construction (e.g., number of components) suitable for providing uniform illumination of the sample 110 through diffusive scattering of the measurement light 114. In this way, the diffusive cavity 102 may ensure that diffusive scattering conditions are maintained during a measurement for the selected sample 110 and potentially for a wide range of samples or conditions. As a result, the step 1102 may include illuminating the sample 110 with under diffusive scattering conditions.

In some embodiments, the method 1100 includes a step 1104 of capturing light exiting at least one of the one or more ports 106 of the diffusive cavity 102 with one or more detectors 116. In some embodiments, the method 1100 includes a step 1106 of receiving detection signals from the one or more detectors 116 indicative of the scattering of the measurement light 114 by the sample 110. In some embodiments, the method 1100 includes a step 1108 of determining one or more time-varying properties of the sample 110 based on the detection signals.

It is contemplated herein that the steps 1104-1108 may be based on signals from any number of detectors 116. In some embodiments, the steps 1104-1108 are based on a single detector 116. In some embodiments, the steps 1104-1108 are based on two or more detectors 116. For example, the step 1108 may include determining one or more time-varying properties of the sample 110 based on the a cross-correlation between detected signals (e.g., two detected signals, or the like).

It is further contemplated herein that the method 1100 may be suitable for measuring time-varying properties of the sample 110 such as, but not limited to, particle size, particle shape, particle concentration, viscosity, or viscoelastic moduli. In this way, and referring generally to FIGS. 1A-11, the systems and methods disclosed herein may further be suitable for a wide range of applications. Further, the use of the diffusive cavity 102 to promote multiple scattering interactions between measurement light 114 and the samples 110 (e.g., to ensure a diffusive scattering regime) may beneficially promote measurements with a high signal to noise ratio and enable measurements of relatively small volumes of a sample 110.

For example, the systems and methods disclosed herein may be useful for measurements of cellular dynamics and fundamental studies of cellular microbiology. For instance, the sample 110 may include one or more cells such that the measured temporal dynamics may be used to quantify various physical or chemical properties of the cells or the interaction of the cells with additional components. As an illustration, the systems and methods disclosed herein may utilize temporal measurements to characterize the impact of external influences on the cells such as, but not limited to, the extracellular environment, applied electromagnetic fields, or the like. As another illustration, the systems and methods disclosed herein may characterize the interaction of the cells with pharmaceuticals or other chemicals based on observable changes to the temporal dynamics. In this way, the systems and methods disclosed herein may be suitable for drug screening.

As another example, the systems and methods disclosed herein may be useful for analytical chemistry measurements. It is noted that the systems and methods disclosed herein may be beneficially provide high-resolution measurements of samples 110 with relatively small volumes and may simplify the procedures for sample preparation which may be elaborate for many traditional techniques. For example, a sample 110 may include two or more compounds such that a reaction between the two or more compounds may be determined based on the temporal dynamics of the sample. As an illustration, measurements of temporal dynamics provided by the systems and methods disclosed herein may be suitable for, but is not limited to, monitoring chemical reactions at small scales, monitoring polymerization dynamics, zeta potential measurements with high precision over a broad concentration of the samples, molecular weight measurements for samples 110 with very different concentrations, high-precision measurements of molecular absorption, measurement of rotational and translational diffusion coefficients of non-spherical nanoparticles, rheological information of the sample, or measurements of any physical and chemical property that would produce minute variations of the refractive index.

In some embodiments, the measurements of temporal dynamics disclosed herein may be combined with additional sample characterization techniques such as, but not limited to, fluorescence or bioluminescence. For example, simultaneous measurements of cell dynamics and fluorescence may enable measurements of quantum yields, fluorescence spectrum, fluorescence lifetime, or the like.

The herein described subject matter sometimes illustrates different components contained within, or connected with, other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “connected” or “coupled” to each other to achieve the desired functionality, and any two components capable of being so associated can also be viewed as being “couplable” to each other to achieve the desired functionality. Specific examples of couplable include but are not limited to physically interactable and/or physically interacting components and/or wirelessly interactable and/or wirelessly interacting components and/or logically interactable and/or logically interacting components.

It is believed that the present disclosure and many of its attendant advantages will be understood by the foregoing description, and it will be apparent that various changes may be made in the form, construction, and arrangement of the components without departing from the disclosed subject matter or without sacrificing all of its material advantages. The form described is merely explanatory, and it is the intention of the following claims to encompass and include such changes. Furthermore, it is to be understood that the invention is defined by the appended claims.

What is claimed:

1. A measurement system comprising:
a diffusive cavity including a reflective internal surface, wherein the diffusive cavity includes one or more ports;
a sample chamber located within the diffusive cavity, the sample chamber configured to hold a sample;
a light source configured to direct measurement light into the diffusive cavity through one of the one or more ports of the diffusive cavity, wherein the diffusive cavity provides uniform illumination of the sample through diffusive reflection of at least one of the measurement light from the light source or scattered measurement light from the sample;
one or more detectors configured to capture light exiting at least one of the one or more ports;
a controller including one or more processors configured to execute program instructions causing the one or more processors to:
receive detection signals from the one or more detectors indicative of the scattering of the measurement light by the sample; and
determine one or more time-varying properties of the sample based on the detection signals.

2. The measurement system of claim 1, wherein the diffusive cavity comprises:
a Cauchy cavity.

3. The measurement system of claim 1, wherein the diffusive cavity comprises:
an integrating sphere.

4. The measurement system of claim 1, wherein the light source comprises:
a coherent light source.

5. The measurement system of claim 1, wherein the light source comprises:
a partially coherent light source.

6. The measurement system of claim 1, wherein the one or more detectors comprise:
two or more detectors.

7. The measurement system of claim 6, wherein determining one or more time-varying properties of the sample based on the detection signals comprises:
generating a cross-correlation between the detection signals, wherein determining one or more time-varying properties of the sample based on the detection signals further comprises:
determining one or more time-varying properties of the sample based on the cross-correlation.

8. The measurement system of claim 6, wherein determining one or more time-varying properties of the sample based on the detection signals comprises:
generating separate detection signals for the two or more detectors, wherein determining one or more time-varying properties of the sample based on the detection signals further comprises:
determining one or more time-varying properties of the sample based on the separate detection signals.

9. The measurement system of claim 6, wherein determining one or more time-varying properties of the sample based on the detection signals further comprises:
determining one or more microscopic characteristic times of the sample based on the detection signals.

10. The measurement system of claim 6, wherein determining one or more time-varying properties of the sample based on the detection signals further comprises:
determining one or more time-varying properties of the sample based on at least one of signal magnitude or spectral composition of the detection signals.

11. The measurement system of claim 1, wherein the one or more detectors comprise:
a single detector.

12. The measurement system of claim 1, wherein at least one of the light source or the one or more detectors are coupled to the diffusive cavity using optical fibers.

13. The measurement system of claim 12, wherein at least one of the one or more detectors comprises:
a single-photon detector.

14. The measurement system of claim 1, wherein at least one of the light source or the one or more detectors are coupled to the diffusive cavity using free-space optical components.

15. The measurement system of claim 14, wherein at least one of the one or more detectors comprises:
a multi-pixel detector, wherein the detection signals from the one multi-pixel detector include one or more images.

16. The measurement system of claim 15, wherein determining one or more time-varying properties of the sample based on the detection signals comprises:
determining the one or more time-varying properties of the sample based on the one or more images.

17. The measurement system of claim 1, wherein the sample chamber comprises:
a transparent vial mounted to the reflective internal surface of the diffusive cavity.

18. The measurement system of claim 1, wherein the sample chamber includes an inlet and an outlet for flow of the sample into or out of the diffusive cavity through at least one of the two or more ports.

19. The measurement system of claim 1, wherein the program instructions further cause the one or more processors to:
determine the one or more time-varying properties of the sample for repeated measurement intervals.

20. The measurement system of claim 1, further comprising:
one or more regulators to regulate environmental conditions of the sample chamber.

21. The measurement system of claim 20, wherein the one or more regulators comprise:
at least one of a temperature regulator, a pressure regulator, a humidity regulator, or an atmospheric composition regulator.

22. The measurement system of claim 20, further comprising:
one or more sensors to monitor the environmental conditions of the sample chamber, wherein the one or more processors are further configured to execute program instructions causing the one or more processors to control the one or more regulators based on data from the one or more sensors.

23. The measurement system of claim 1, wherein the one or more time-varying properties of the sample comprise:
at least one of a characteristic time or a cross-correlation.

24. The measurement system of claim 1, wherein the one or more time-varying properties of the sample comprise:
at least one of particle size, particle shape, particle concentration, viscosity, or viscoelastic moduli.

25. The measurement system of claim 1, wherein the one or more time-varying properties of the sample comprise:
at least one of concentration, molecular weight, zeta potential, molecular absorption, a diffusion, or refractive index.

26. The measurement system of claim 1, wherein the sample in the sample chamber includes one or more cells.

27. The measurement system of claim 26, wherein the program instructions further cause the one or more processors to:
quantify at least one of one or more physical properties or one or more chemical properties of the one or more cells based on the time-varying properties of the sample.

28. The measurement system of claim 26, wherein the sample in the sample chamber further includes one or more additional materials, wherein the program instructions further cause the one or more processors to:
determine one or more parameters of a reaction between the one or more cells and the one or more additional materials.

29. The measurement system of claim 28, wherein the one or more additional materials include one or more pharmaceuticals.

30. The measurement system of claim 1, wherein the sample in the sample chamber includes two or more compounds, wherein the program instructions further cause the one or more processors to:
determine one or more parameters of a reaction between the two or more compounds.

31. The measurement system of claim 1, further comprising:
one or more field generators to expose the sample to one or more external fields when determining the one or more time-varying properties of the sample based on the detection signals.

32. The measurement system of claim 31, wherein the one or more field generators comprise:
at least one of an electric field generator, a magnetic field generator, or an acoustic field generator.

33. The measurement system of claim 31, wherein the one or more external fields are static.

34. The measurement system of claim 31, wherein the one or more external fields are time-varying.

35. A measurement method comprising:
directing measurement light into a diffusive cavity through one of one or more ports of the diffusive cavity, wherein the diffusive cavity includes a reflective internal surface, wherein the diffusive cavity provides uniform illumination of a sample in the diffusive cavity through diffusive reflection of at least one of the measurement light or scattered measurement light from the sample;
capturing light exiting at least one of the one or more ports of the diffusive cavity with one or more detectors;
receiving detection signals from the one or more detectors indicative of the scattering of the measurement light by the sample; and
determining one or more time-varying properties of the sample based on the detection signals.

36. The measurement method of claim 35, wherein determining the one or more time-varying properties of the sample based on the detection signals comprises:
determining at least one of a characteristic time or a cross-correlation based on the detection signals.

37. The measurement method of claim 35, wherein determining the one or more time-varying properties of the sample based on the detection signals comprises:
determining at least one of particle size, particle shape, particle concentration, viscosity, viscoelastic moduli, concentration, molecular weight, zeta potential, molecular absorption, a diffusion, or refractive index based on the detection signals.

38. The measurement method of claim 35, wherein the one or more detectors comprise:
two or more detectors.

39. The measurement method of claim 38, wherein determining one or more time-varying properties of the sample based on the detection signals comprises:
generating a cross-correlation between the detection signals, wherein determining one or more time-varying properties of the sample based on the detection signals further comprises:
determining the one or more time-varying properties of the sample based on the cross-correlation.

40. The measurement method of claim 38, wherein determining one or more time-varying properties of the sample based on the detection signals comprises:
generating separate detection signals for the two or more detectors, wherein determining one or more time-varying properties of the sample based on the detection signals further comprises:
determining the one or more time-varying properties of the sample based on the separate detection signals for the two or more detectors.

41. The measurement method of claim 35, wherein the one or more detectors comprise:
a single detector.

42. The measurement method of claim 35, further comprising:
determining the one or more time-varying properties of the sample for repeated measurement intervals.
